(19) United States
(12) Patent Application Publication
(10) Pub. No.: US 2006/0178573 A1
(43) Pub. Date: Aug. 10, 2006

(54) System and method for piercing dermal tissue

(76) Inventors: Mahyar Z Kermani, Pleasanton, CA (US); Borzu Sohrab, Los Altos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/511,495

(22) PCT Filed: Feb. 3, 2004
(86) PCT No.: PCT/US04/03142

§ 371 Date: Jan. 3, 2006

Related U.S. Application Data
(60) Provisional application No. 60/452,409, filed on Mar. 6, 2003.

(51) Int. Cl. A61B 5/05

(52) U.S. Cl. 600/347

(57) ABSTRACT

A system for piercing dermal tissue includes a skin-piercing element (e.g., an integrated micro-needle and biosensor medical device), at least one electrical contact (e.g., an electrical skin contact) and a meter configured for measuring an electrical characteristic (e.g., resistance and/or impedance) existent between the skin-piercing element and the electrical contact(s) when the system is in use. The electrical contact(s) can be integrated with a pressure/contact ring of the meter to provide a compact and inexpensive system compatible with integrated micro-needle and biosensor medical devices. Also, a method for piercing dermal tissue that includes contacting dermal tissue (e.g., skin) with at least one electrical contact and inserting a skin-piercing element into the dermal tissue while measuring an electrical characteristic existent between the skin-piercing element and the electrical contact(s).

### BACKGROUND OF INVENTION

1. Field of the Invention

The present invention relates, in general, to medical devices and, in particular, to medical devices and associated methods for piercing dermal tissue.

2. Description of the Related Art

A variety of medical procedures (e.g., the sampling of whole blood for glucose or other analyte monitoring) involve the penetration of dermal tissue (e.g., skin) by a skin-piercing element (e.g., a lancet or micro-needle). During such procedures, the depth, stability and duration of dermal tissue penetration by the skin-piercing element can be important factors in determining the outcome of the procedure. For example, insufficient penetration depth can be an erroneous condition that results in an unsatisfactory outcome for certain medical procedures.

Recently, micro-needles and biosensors (e.g., electrochemical-based and photometric-based biosensors) have been integrated into a single medical device. These integrated medical devices can be employed, along with an associated meter, to monitor various analytes, including glucose. Depending on the situation, biosensors can be designed to monitor analytes in an episodic single-use format, semi-continuous format, or continuous format. The integration of a micro-needle and biosensor simplifies a monitoring procedure by eliminating the need for a user to coordinate the extraction of a sample from a sample site with the subsequent transfer of that sample to a biosensor. This simplification, in combination with a small micro-needle and a small sample volume, also reduces pain and enables a rapid recovery of the sample site.

The use of integrated micro-needle and biosensor medical devices and their associated meters can, however, decrease the ability of a user to detect deleterious conditions, such as erroneous conditions related to insufficient or unstable skin penetration during the required sample extraction and transfer residence time. Such erroneous conditions can, for example, result in the extraction and transfer of a sample with an insufficient volume for accurate measurement of an analyte therein. Furthermore, in some circumstances, it can be important that a micro-needle's penetration be stable for an extended period of time (e.g., several hours or days). Such stability is important, for example, during continuous monitoring where interruptions in micro-needle penetration can introduce air bubbles into a fluidic pathway of a medical device. Additionally, instability could interrupt an electrical circuit needed for the electrochemical measurement of analyte when the micro-needle is also used as a reference or working electrode.

Still needed in the field, therefore, are medical devices and associated methods that can detect and/or provide an indication of penetration depth, sample extraction and transfer residence time and/or stability during the piercing of dermal tissue. In addition, the systems and methods should be compatible with integrated micro-needle and biosensor medical devices and their associated meters.

### SUMMARY OF INVENTION

Embodiments of systems and methods for piercing dermal tissue according to the present invention can detect and/or provide an indication of penetration depth, sample extraction and transfer residence time and/or stability during piercing. In addition, the systems and methods are compatible with integrated micro-needle and biosensor medical devices and their associated meters.

A system for piercing dermal tissue according to an exemplary embodiment of the present invention includes a skin-piercing element (e.g., an integrated micro-needle and biosensor medical device), at least one electrical contact (e.g., an electrical skin contact) and a meter configured for measuring an electrical characteristic (e.g., resistance and/or impedance) existent between the skin-piercing element and the electrical contact(s) when the system is in use. The electrical contact(s) can, for example, be an electrical skin contact that is integrated with a pressure/contact ring of the meter. Integration of the electrical contact and pressure/contact ring provides a compact and inexpensive system compatible with integrated micro-needle and biosensor medical devices.

The ability of systems according to the present invention to detect and indicate penetration depth, duration (i.e., residence time) and/or stability is based on the concept that the measured electrical characteristic between the electrical contact and the skin-piercing element is indicative of the aforementioned depth, stability and/or duration. For example, it has been determined that the impedance between a skin-piercing element (e.g., a micro-needle) and one or more electrical skin contacts is indicative of dermal tissue penetration depth by the skin-piercing element. Furthermore, changes in such impedance can be indicative of penetration stability and/or duration.

In embodiments of systems according to the present invention, the impedance (or other electrical characteristic) is measured by techniques that involve, for example, applying a safe electrical potential between the electrical contact and the skin-piercing element while the system is in use.

Also provided is a method for piercing dermal tissue that includes contacting dermal tissue (e.g., skin) with at least one electrical contact and inserting a skin-piercing element into the dermal tissue while measuring an electrical characteristic existent between the skin-piercing element and the electrical contact(s).

### BRIEF DESCRIPTION OF DRAWINGS

A better understanding of the features and advantages of the present invention will be obtained by reference to the following detailed description that sets forth illustrative embodiments, in which the principles of the invention are utilized, and the accompanying drawings, of which:

FIG. 1 is a simplified depiction of dermal tissue and a system for piercing dermal tissue according to an exemplary embodiment of the present invention wherein a skin-piercing element of the system is out of contact with the dermal tissue;

FIG. 2 is a top perspective exploded view of an integrated micro-needle and biosensor medical device (also referred to as an electrochemical test strip) that can be employed in embodiments of systems according the present invention;

FIG. 3 is a bottom perspective exploded view of the integrated micro-needle and biosensor medical device of FIG. 2;

FIG. 4 is a top perspective view of the integrated micro-needle and biosensor medical device of FIG. 2;

FIG. 5 is a simplified depiction of a system according to another embodiment of the present invention that includes skin-piercing element (in the form of an integrated micro-needle and biosensor medical device), an electrical skin contact (integrated with a pressure/contact ring) and a meter;

FIG. 6 is a simplified electrical schematic and block diagram depiction of the system of FIG. 1, including various components of the meter;

FIG. 7 is a simplified depiction of the system of FIG. 1, wherein the skin-piercing element is in non-penetrating contact with the dermal tissue;

FIG. 8 is a simplified depiction of the system of FIG. 1, wherein the skin-piercing element has penetrated the dermal tissue;

FIG. 9 is a simplified depiction of dermal tissue and a system for piercing dermal tissue according to yet another embodiment of the present invention, wherein a skin-piercing element of the system is out of contact with the dermal tissue;

FIG. 10 is a simplified depiction of the system of FIG. 9, wherein the skin-piercing element is in non-penetrating contact with the dermal tissue;

FIG. 11 is a simplified depiction of the system of FIG. 1, wherein the skin-piercing element has penetrated the dermal tissue;

FIG. 12 is a simplified electrical schematic and block diagram depiction of the system of FIG. 9, including various components of the meter; and

FIG. 13 is a flow chart illustrating a sequence of steps in a process according to an exemplary embodiment of the present invention.

### DETAILED DESCRIPTION OF THE INVENTION

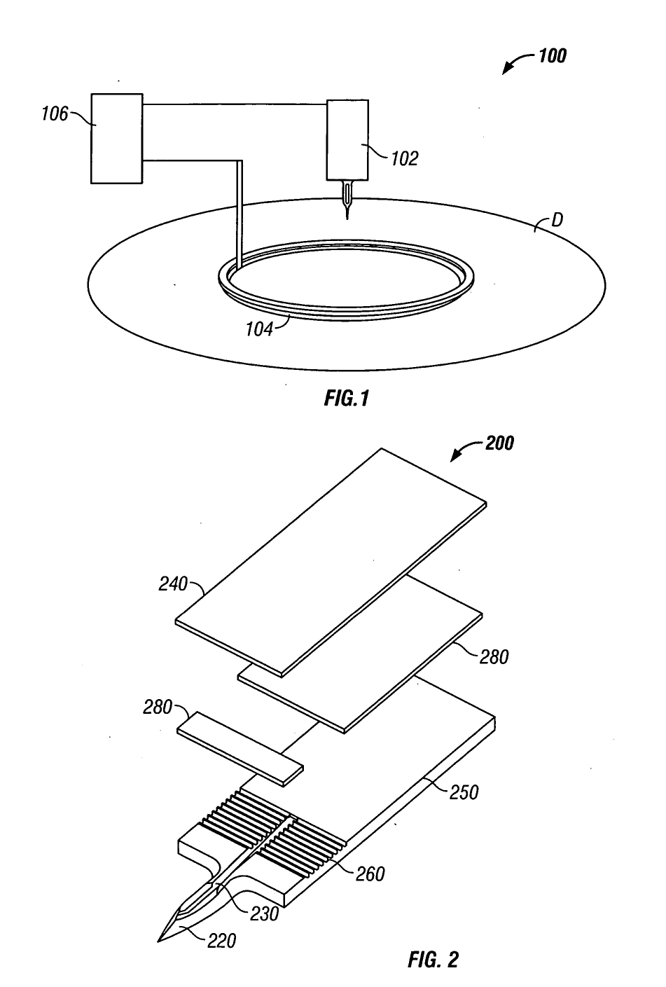

FIG. 1 is simplified depiction of a system 100 for piercing dermal tissue D. System 100 includes a skin-piercing element 102, at least one electrical contact 104 and a meter 106 configured for measuring an electrical characteristic (e.g., resistance and/or impedance) that exists between the skin-piercing element 102 and the electrical contact(s) 104 when system 100 is in use.

Skin-piercing element 102 can be any suitable skin-piercing element known to one skilled in art including, but not limited to, lancets, micro-needles and micro-needles that have been integrated with a biosensor to form an integrated micro-needle and biosensor medical device. Those skilled in the art will recognize that micro-needles serving as skin-piercing elements can take any suitable form including, but not limited to, those described in U.S. patent application Ser. No. 09/919,981 (filed on Aug. 1, 2001), Ser. No. 09/923,093 (filed on Aug. 6, 2001), Ser. No. 10/143,399 (filed on May 9, 2002), Ser. No. 10/143,127 (filed on May 9, 2002), and Ser. No. 10/143,422 (filed on May 9, 2002), as well as PCT Application WO 01/49507A1, each of which is hereby incorporated in full by reference.

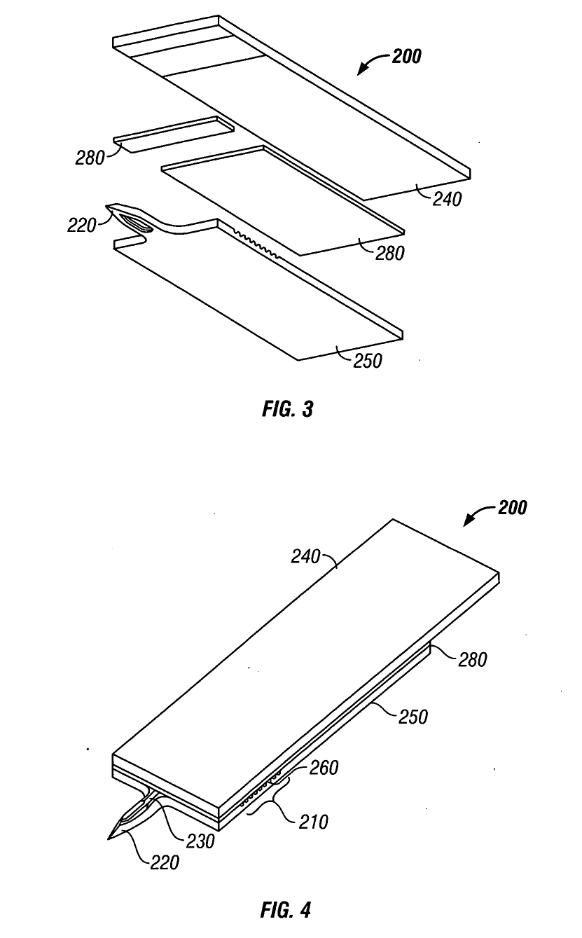

FIGS. 2 through 4 depict an integrated micro-needle and biosensor medical device 200 (also referred to as an electrochemical test strip) that can be beneficially employed as the skin-piercing element in embodiments of systems according to the present invention. Medical device 200 includes an electrochemical cell 210, an integrated micro-needle 220 and an integrated capillary channel 230. Electrochemical cell 210 includes a working electrode 240, a reference electrode 250, spreading grooves 260 and a reagent composition (not illustrated). Alternatively, medical device 200 can be configured without spreading grooves 260.

Working electrode 240 and reference electrode 250 are oppositely spaced apart by divided spacer layer 280, as illustrated in FIGS. 2 through 4. Divided spacer layer 280 serves to define, along with working electrode 240 and reference electrode 250, the boundaries of electrochemical cell 210. Working electrode 240 and reference electrode 250 can be formed of any suitable material. The reagent composition includes, for example, a redox enzyme and a redox couple. The reagent composition can be deposited on one or more of the reference and working electrode by any conventional technique including, for example, screen printing, spraying, ink jetting and slot coating techniques.

Integrated micro-needle 220 is adapted for obtaining (extracting) a whole blood sample from a user and introducing (transferring) the whole blood sample into the electrochemical cell 210 via integrated capillary channel 230. Once introduced into the electrochemical cell 210, the whole blood sample distributes evenly across spreading grooves 260. Integrated micro-needle 220 can be adapted for obtaining (extracting) and introducing (transferring) an interstitial fluid sample rather than a whole blood sample.

Integrated micro-needle 210 can be manufactured of any suitable material including, for example, a plastic or stainless steel material that has been sputtered or plated with a noble metal (e.g., gold, palladium, iridium or platinum). The shape, dimensions, surface features of the integrated micro-needle, as well as the working penetration depth of the micro-needle into a user's epidermal/dermal skin layer (e.g., dermal tissue), are adapted to minimize any pain associated with obtaining a whole blood sample from the user.

During use of medical device 200 (also referred to as an electrochemical test strip), a sample (such as, whole blood) is introduced into electrochemical cell 210 via integrated capillary channel 230 and is distributed evenly within electrochemical cell 210 by spreading grooves 260 when a user's skin is punctured (i.e., penetrated) by integrated micro-needle 220. In FIGS. 2 through 4, integrated micro-needle 220 is illustrated as integrated with reference electrode 250. However, one skilled in the art will recognize that integrated micro-needle 220 can be alternatively integrated with working electrode 240.

Although medical device 200 has a working electrode and a reference electrode that are configured in an opposing faced orientation and in separate planes, one skilled in the art will recognize that medical devices wherein a working electrode and a reference electrode are configured in the same plane can also be beneficially employed as the skin-piercing element in embodiments of systems according to the present invention. Such medical devices are described, for example, in U.S. Pat. No. 5,708,247, U.S. Pat. No. 5,951,836, U.S. Pat. No. 6,241,862, and PCT Applications WO 01/67099, WO 01/73124, and WO 01/73109, each of which is hereby incorporated in full by reference.

It should be noted that one skilled in the art would recognize that a photometric-based test strip, instead of an electrochemical-based test strip, can be employed in alternative embodiments of this invention. Examples of such photometric strips are described in U.S. patent application Ser. No. 09/919,981 (filed on Aug. 1, 2001), Ser. No. 09/923,093 (filed on Aug. 6, 2001), Ser. No. 10/143,399 (filed on May 9, 2002), Ser. No. 10/143,127 (filed on May 9, 2002) and Ser. No. 10/143,422 (filed on May 9, 2002), each of which is hereby incorporated in full by reference.

Referring again to FIG. 1, electrical contact 104 can be any suitable electrical contact known to one skilled in the art. In the embodiment of FIG. 1, electrical contact 104 has a circular shape and is an electrical skin contact adapted for making electrical contact with the outer skin layer of dermal tissue D. Electrical contact 104 includes an outer electrically conductive layer that, during use, is in contact with the outer skin layer. Such a conductive layer can be applied by conventional processes such as electro-less plating, sputtering, evaporation and screen printing.

One skilled in the art will recognize that electrical contact 104 can be formed of a conductive material in order to enable the ready measurement of an electrical characteristic existing between the skin-piercing element and the electrical contact. Electrical contact 104 can be formed from any suitable electrically conductive material, for example, a polarizable electrode material such as Au, Pt, carbon, doped tin oxide and Pd, conductive polyurethane, or a non-polarizable electrode material such as Ag/AgCl.

In order to provide a system that is compact and compatible with integrated micro-needle and biosensor medical devices and their associated meters, it can be beneficial to integrate the electrical contact with a pressure/contact ring of such meters. The integrated electrical contact and pressure/contact ring can then, for example, be electrically connected to an impedance measuring device located within a housing of the meter.

In the circumstance that the electrical contact and pressure/contact ring have been integrated, electrical contact 104 can be applied to dermal tissue D at a pressure of, for example, 0.5 to 1.5 pounds to facilitate the egress of bodily fluids. An integrated electrical contact and pressure/contact ring can have, for example, a diameter in the range of from 2 mm to 10 mm. Such an integrated electrical contact and pressure/contact ring helps facilitate the milking of fluid egress from the dermal tissue target site and is adapted for monitoring an electrical characteristic to ensure sufficient skin penetration, penetration stability and/or a sufficient residence time (duration) of the skin-piercing element within the dermal tissue.

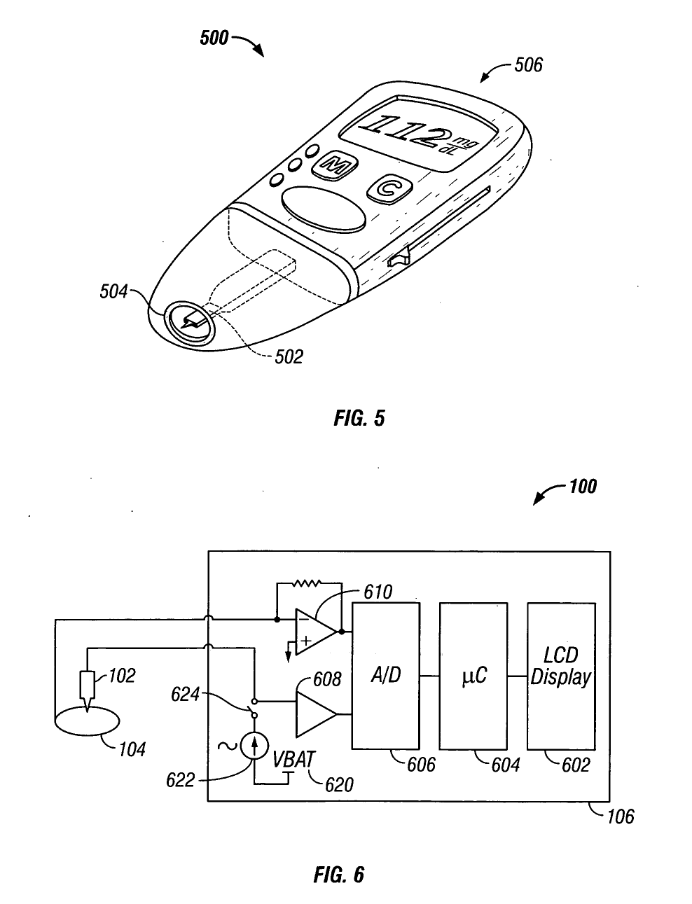

The optional integration of the electrical contact ring and a pressure/contact ring is illustrated in FIG. 5. FIG. 5 depicts an exemplary embodiment of a system 500 for piercing dermal tissue. System 500 includes a skin-piercing element 502 (i.e., an integrated micro-needle and electrochemical test strip), an integrated electrical contact and pressure/contact ring 504 and a meter 506 for measuring impedance between the skin-piercing element 502 and the integrated electrical contact and pressure/contact ring 504 to ascertain whether sufficient skin penetration has been achieved. The meter depicted in FIG. 5 is a novel modification of the meter described in US2002/0168290, entitled “Physiological Sample Collection Devices and Methods of Using the Same,” which is hereby incorporated in full by reference. Once apprised of the present disclosure, one skilled in the art will recognize that a variety of pressure/contact rings can be integrated with an electrical contact for use in embodiments of the present invention. Examples of such pressure/contact rings are described in U.S. Patent Application Publication No. 2002/0016606, U.S. Pat. No. 6,283,982, and PCT Application WO 02/078533A2, each of which are hereby incorporated in full by reference.

Referring again to FIG. 1, meter 106 can be any suitable meter known to one skilled in the art that is configured for measuring an electrical characteristic (e.g., resistance and/or impedance) existent between the skin-piercing element 102 and the at least one electrical contact 104 when system 100 is in use. Meter 106 can measure the electrical characteristic (e.g., impedance) by, for example, applying a safe potential and/or current (which will be described further, in terms of current amplitude and frequency ranges, below) between the skin-piercing element and the electrical contact when the system is in use. For example, the electrical characteristic can be measured when the skin-piercing element approaches, makes non-penetrating contact with, penetrates (e.g., pierces) and is retracted from the dermal tissue. Furthermore, the electrical characteristic can be measured continuously throughout the aforementioned use. In this exemplary circumstance, dermal tissue penetration by the skin-piercing element can be detected based on a significant decrease in an electrical characteristic (e.g., impedance), retraction of the skin-piercing element from the dermal tissue can be detected based on a significant increase in the electrical characteristic, the duration of penetration can be determined as the time between penetration and retraction, and stability can be detected based on fluctuations in the electrical characteristic. The frequency at which the potential and/or current is applied can be varied to minimize dependence on variations in skin type and condition.

FIG. 6 serves to further illustrate a suitable meter for use in system 100. In the embodiment of FIG. 6, meter 106 includes an LCD display 602, micro-controller (μC) 604, an analog-to-digital converter (A/D) 606, an amplifier 608, current-to-voltage converter 610, battery (VBAT) 620, an AC current source 622 and a switch 624. Meter 106 is adapted to electronically interface with skin-piercing element 102 and electrical contact 104. When switch 624 is closed (i.e., on), the meter 106 applies an AC current waveform between skin-piercing element 102 and electrical contact 104 for the purpose of measuring impedance therebetween. By measuring the current (I) and the voltage (V) across the skin-piercing element and electrical contact, the impedance (Z) can be calculated using Ohm's law:
Z=V/I
If so desired, either resistance or capacitance can also be determined from the impedance value.

It is beneficial if the amplitude of the current source is limited to values that can not be sensed by a user (e.g., less than 10 mA) but large enough (e.g., more than 1 mA) to create a good signal to noise ratio. In an exemplary embodiment of this invention, the current frequency is between 10 KHz to 1 MHz, where the low end of the frequency range prevents user discomfort and the high end of the frequency range minimizes stray capacitance from being measured.

The measurement of impedance using a measured AC voltage and current traditionally requires a fast A/D converter and other relatively expensive electrical components. However, systems according to the present invention can also provide for impedance measurements using relatively inexpensive techniques described in pending applications U.S. patent application Ser. No. 10/020,169 (filed on Dec. 12, 2001) and U.S. patent application Ser. No. 09/988,495 (filed on Nov. 20, 2001), each of which is hereby incorporated by reference.

FIG. 1 depicts a spatial relationship of skin-piercing element 102, dermal tissue D and electrical contact 104 for the circumstance that the skin-piercing element is out of contact with dermal tissue D (i.e., is not in contact with the skin layer of dermal tissue D). For this spatial relationship, the impedance between the skin-piercing element and the electrical contact (which is in contact with the outer skin layer of dermal tissue D) is typically greater than 10 MΩ. It should be noted, however, that the impedance value can vary depending on the type of electronics used in the meter and the magnitude of any leakage current.

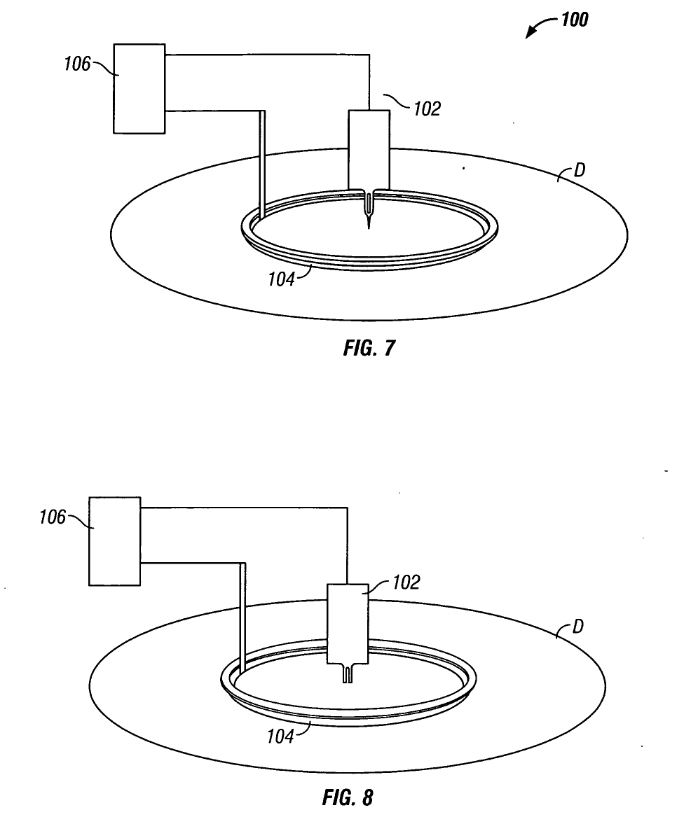

FIG. 7 is a schematic showing the spatial relationship of skin-piercing element 102, dermal tissue D and electrical contact 104, for the circumstance that the skin-piercing element is in non-penetrating contact with dermal tissue D at the center point of the circle formed by electrical contact 104. For this spatial relationship, the impedance between the skin-piercing element 102 and the electrical contact 104 is typically, for example, in the range between 15 kΩ to approximately 1 MΩ.

FIG. 8 is a schematic showing the spatial relationship of skin-piercing element 102, dermal tissue D and electrical contact 104, for the circumstance that the skin-piercing element has penetrated dermal tissue D at the center point of the circle formed by electrical contact 104. For this spatial relationship, the impedance between skin-piercing element 102 and the electrical contact 104 is low, typically no more than 10% of the impedance for the circumstance that the skin-piercing element is in non-penetrating contact with dermal tissue D. It is postulated, without being bound, that this large change in impedance is due to the majority of the impedance of skin being in the outer layer or epidermis and that penetration of the skin-piercing element into the dermal tissue beyond the outer layer reduces impedance significantly.

Based on the discussion above, it is evident that the measurement of the impedance between the skin-piercing element and the electrical contact while the system is in use provides an indication of skin penetration, as well as, the stability of this penetration. In other words, the system's meter can detect penetration, penetration stability and penetration duration (i.e., sample extraction and transfer residence time) by measuring the impedance (or resistance) between the skin-piercing element and the electrical contact. When the skin-piercing element penetrates into the dermal tissue, the resistance or impedance will exhibit a significant change.

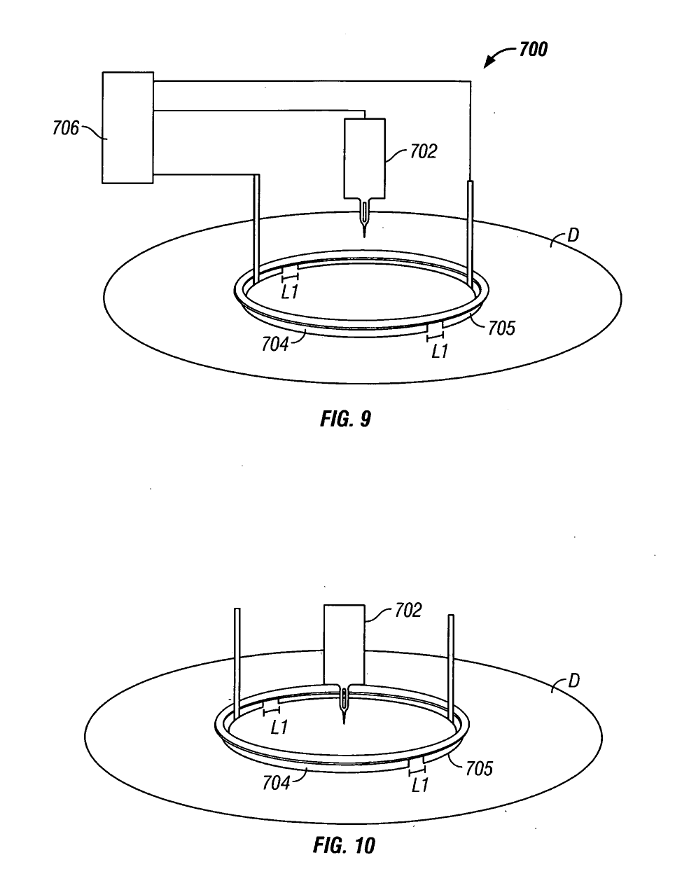

In order to lessen any impact of skin resistance differences on electrical characteristic measurements, a plurality of electrical contacts can be employed. In this circumstance, an additional measurement of the electrical characteristic between the electrical contacts can be used to normalize subsequent measurements between the electrical contacts and the skin-piercing element. Although any number of electrical contacts can be employed, for the sake of simplicity, system 700 of FIG. 9 for piercing dermal tissue D is depicted as including two electrical contacts. System 700 includes a skin-piercing element 702, a first electrical contact 704, a second electrical contact 705 and a meter 706 configured for measuring an electrical characteristic (e.g., resistance and/or impedance) that exists between the skin-piercing element 702 and both of the first and second electrical contacts 704 and 705. The use of a first and a second electrical contact allows the detection of penetration to be less dependent on skin type and condition by providing for differential electrical characteristic measurements between the two electrical contacts.

Dermal tissue impedance can vary due to humidity of the environment or sweating caused by high temperature or exercise. In the embodiment of FIGS. 9 through 11, two additional impedance measurements which can be monitored are those between skin-piercing element 702 and first electrical contact 704, and between skin-piercing element 702 and second electrical contact 705. By averaging impedance values measured between the skin-piercing element and both the first and second electrical contacts, the ability to accurately detect dermal tissue penetration is improved. In addition, measurements of the impedance between the skin-piercing element and both the first and second contacts can be a basis for a determination as to whether or not uniform pressure has been applied to the first and second electrical contacts. Furthermore, the determination of whether or not uniform pressure has been applied can mitigate the risk of positioning the skin-piercing element such that it penetrates the dermal tissue in a non-perpendicular manner. Although the embodiment of FIGS. 9 through 11 employs two electrical contacts, it should be appreciated that one skilled in the art could also employ more than two electrical contacts and, thereby, improve resolution when determining if a skin-piercing element is being applied in a perpendicular manner.

Furthermore, the measured impedance between the first and second electrical contacts can be used to normalize impedance values measured between the first electrical contact and the skin-piercing element, as well as between the second electrical contact and the skin-piercing element. The normalized impedance R can be calculated as the following:
R=Rₙ/R_{b}
where:
Rₙ is the impedance between the skin-piercing element and either the first or the second electrical contact or, alternatively, the average of the impedance between the skin-piercing element and each of the first and second electrical contacts; and
R_{b} is the impedance measurement between the first and second electrical contacts.

FIG. 9 depicts a spatial relationship of skin-piercing element 702, dermal tissue D, and first and second electrical contacts 704, 705 for the circumstance that the skin-piercing element is out of contact with dermal tissue D (i.e., is not in contact with the skin layer of dermal tissue D). In system 700, first and second electrical contacts 704, 705 are insulated from one another and separated by a distance L1, as illustrated in FIGS. 9 through 11. Distance L1 is typically in the range of 0.5 mm to 2 mm, when L1 is defined as the closest gap between the first and second electrical contacts 704, 705. For the spatial relationship of FIG. 9, the impedance between the skin-piercing element 702 and the first electrical contact 704 and between the skin-piercing element 702 and the second electrical contact 705 is typically greater than 10 MΩ. Additionally, the impedance between first electrical contact 704 and the second electrical contact is a finite value typically in the range between 15 kΩ to approximately 1 MΩ.

FIG. 10 is a schematic showing the spatial relationship of skin-piercing element 702, dermal tissue D and first and second electrical contacts 704 and 705, for the circumstance that the skin-piercing element is in non-penetrating contact with dermal tissue D. For this spatial relationship, the impedance between the skin-piercing element 702 and the first electrical contact 704 and between the skin-piercing element 702 and the second electrical contact 705 is typically, for example, in the range between 15 kΩ to approximately 1 MΩ. Additionally, the impedance between first electrical contact 704 and the second electrical contact 705 is a finite value typically in the range between 15 kΩ to approximately 1 MΩ.

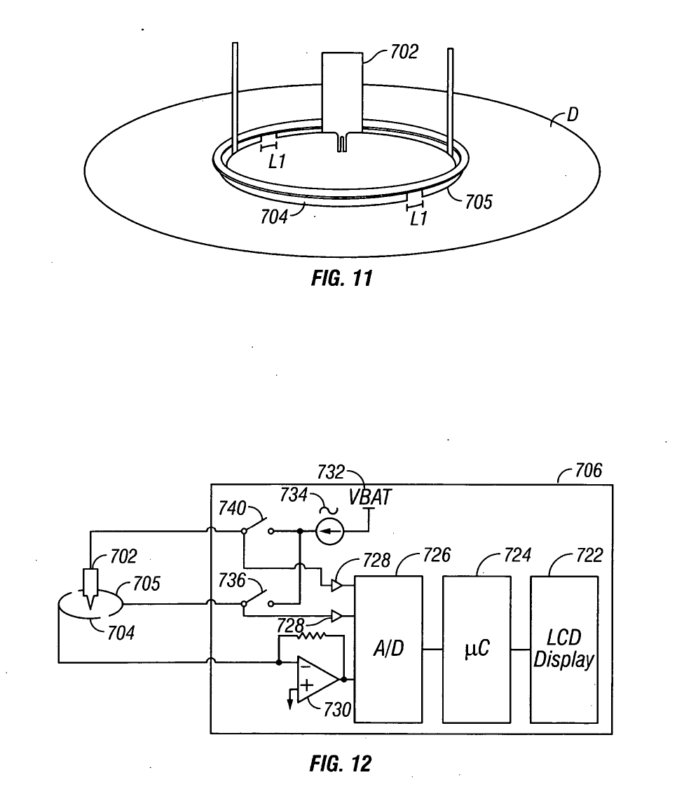

FIG. 11 is a schematic showing the spatial relationship of skin-piercing element 702, dermal tissue D and first and second electrical contacts 704 and 705, for the circumstance that the skin-piercing element has penetrated dermal tissue D. For this spatial relationship, the impedance between skin-piercing element 102 and either of first and second electrical contacts 704 and 705 is low, typically no more than 10% of the impedance for the circumstance that the skin-piercing element is in non-penetrating contact with dermal tissue D. Additionally, the impedance between first electrical contact 704 and second electrical contact 705 is a finite value typically in the range between 15 kΩ to approximately 1 MΩ.

FIG. 12 serves to further illustrate a suitable meter 706 for use in system 700 that includes suitable electronic components for measuring an electrical characteristic (i.e., impedance) between skin-piercing element 702 and either of first and second electrical contacts 704 and 705. Meter 706 is depicted in FIG. 12 as including an LCD display 722, a micro-controller (μC) 724, an analog-to-digital converter (A/D) 726, amplifiers 728, current-to-voltage converter 730, battery (VBAT) 732, an AC current source 734, and a first switch 736 and a second switch 740. Meter 706 is operatively connected with skin-piercing element 702, first electrical contact 704 and second electrical contact 705. When first switch 736 is closed (i.e., on) and second switch 740 is open (i.e., off), the meter applies an AC current waveform between second electrical contact 705 and first electrical contact 704 for the purpose of measuring impedance therebetween. When first switch 736 is open and second switch 740 is closed, the meter applies an AC current waveform between skin-piercing element 702 and first electrical contact 704 for the purpose of measuring impedance therebetween. When both first switch 736 and second switch 740 are open, the meter 706 can be used, for example, to measure and output a glucose value.

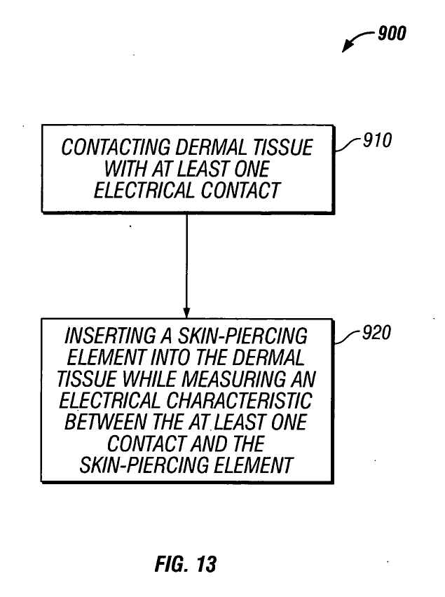

FIG. 13 is a flow chart illustrating a sequence of steps in a process 900 according to an exemplary embodiment of the present invention. Process 900 includes contacting dermal tissue with at least one electrical contact, as set forth in step 910 and inserting a skin-piercing element (e.g., an integrated micro-needle and biosensor) into the dermal tissue, as set forth in step 920. During the insertion, an electrical characteristic (e.g., resistance or impedance) existent between the skin-piercing element and the electrical contact(s) is measured. The concept underlying process 900 is that the changes in the measured electrical characteristic can indicate a sufficient depth of dermal tissue penetration and/or a sufficient sample extraction and transfer residence time (duration) and/or the stability of skin-piercing element within the dermal tissue.

If desired, process 900 can also includes presenting a user with an indicator (e.g., a visual or auditory indicator) of a dermal tissue penetration depth of the skin-piercing element, an indicator of a dermal tissue penetration stability of the skin-piercing element, and/or an indicator of dermal tissue penetration duration (i.e., sample extraction and transfer residence time) of the skin-piercing element, with said indicator being based on the measured electrical characteristic.

It should be understood that various alternatives to the embodiments of the invention described herein may be employed in practicing the invention. It is intended that the following claims define the scope of the invention and that structures and methods within the scope of these claims and their equivalents be covered thereby.

What is claimed is:

1. A system for piercing dermal tissue, the system comprising
a skin-piercing element;
at least one electrical contact; and
a meter configured for measuring an electrical characteristic existent between the skin piercing element and the at least one electrical contact when the system is in use.

2. The system of claim 1, wherein the at least one electrical contact is an electrical skin contact.

3. The system of claim 1, wherein the meter is configured to measure an electrical characteristic between the skin-piercing element and the at least one electrical contact that is indicative of dermal tissue penetration by the skin-piercing element.

4. The system of claim 1, wherein the meter is configured to measure an electrical characteristic between the skin-piercing element and the at least one electrical contact that is indicative of a stability of dermal tissue penetration by the skin-piercing element.

5. The system of claim 1, wherein the meter is configured to measure an electrical characteristic between the skin-piercing element and the at least one electrical contact that is indicative of dermal tissue penetration residence time by the skin-piercing element.

6. The system of claim 1, wherein the electrical characteristic is the electrical resistance between the skin-piercing element and the at least one electrical contact.

7. The system of claim 1, wherein the electrical characteristic is the electrical impedance between the skin-piercing element and the at least one electrical contact.

8. The system of claim 1, wherein the at least one electrical contact includes a first electrical contact and a second electrical contact.

9. The system of claim 8, wherein the meter is further configured for measuring an electrical characteristic existent between the first and second electrical contacts.

10. The system of claim 1, wherein the meter includes a pressure/contact ring and the at least one electrical contact is integrated with the pressure/contact ring.

11. The system of claim 1, wherein the skin-piercing element is a micro-needle.

12. The system of claim 11, wherein the micro-needle is a component of an integrated micro-needle and biosensor medical device.

13. A system for piercing dermal tissue, the system comprising
a skin-piercing element;
a first electrical contact;
a second electrical contact; and
a meter configured for measuring an electrical characteristic existent between the skin piercing element and the first and second electrical contacts when the system is in use.

14. The system of claim 13, wherein the electrical characteristic is the electrical impedance between the skin-piercing element and both of the first and second electrical contacts.

15. The system of claim 13, wherein the meter includes a pressure/contact ring and the first and second electrical contacts are integrated with the pressure/contact ring.

16. The system of claim 13, wherein the skin-piercing element is a micro-needle.

17. The system of claim 16, wherein the micro-needle is a component of an integrated micro-needle and biosensor medical device.

18. The system of claim 13, wherein the first electrical contact is a first electrical skin contact and the second electrical contacts is a second electrical skin contact.

19. A method for piercing dermal tissue comprising:
contacting dermal tissue with at least one electrical contact; and
inserting a skin-piercing element into the dermal tissue while measuring an electrical characteristic existent between the skin-piercing element and the at least one electrical contact, thereby penetrating the dermal tissue.

20. The method of claim 19 further including the step of presenting a user with an indicator of a dermal tissue penetration depth of the skin-piercing element, said indicator being based on the measured electrical characteristic.

21. The method of claim 19 further including the step of presenting a user with an indicator of a dermal tissue penetration stability of the skin-piercing element, said indicator being based on the measured electrical characteristic.

22. The method of claim 19 further including the step of presenting a user with an indicator of dermal tissue penetration residence time of the skin-piercing element, said indicator being based on the measured electrical characteristic.

23. The method of claim 19, wherein the inserting step includes inserting a micro-needle skin-piercing element.

24. The method of claim 19, wherein the inserting step includes inserting a micro-needle of an integrated micron-needle and biosensor medical device.

25. The method of claim 19, wherein the inserting step further involves measuring the electrical characteristic prior to contact between the skin-piercing element and the dermal tissue, when the skin-piercing element has contacted the dermal tissue and when the skin-piercing element has penetrated the dermal tissue.

26. The method of claim 19, wherein the measuring is accomplished by applying a current in the range of 1 mA to 10 mA.

27. The method of claim 19, wherein the measuring is accomplished using a potential frequency in the range of 10 KHz to 1 MHz, where the low end of the frequency prevents user discomfort and the high end of the frequency minimizes stray capacitance from being measured.
